(19) United States
(12) Patent Application Publication
(10) Pub. No.: US 2019/0179040 A1
(43) Pub. Date: Jun. 13, 2019

(54) INTEGRATED MULTI-SLICE X-RAY DETECTOR FOR IN-LINE COMPUTED TOMOGRAPHY

(71) Applicant: X-Scan Imaging Corporation, San Jose, CA (US)

(72) Inventors: Nguyen Phuoc Luu, Los Gatos, CA (US); Chinlee Wang, Saratoga, CA (US)

(73) Assignee: X-Scan Imaging Corporation

(21) Appl. No.: 16/215,418

(22) Filed: Dec. 10, 2018

Related U.S. Application Data
(60) Provisional application No. 62/596,136, filed on Dec. 8, 2017.

(51) Int. Cl. G01T 1/20; A61B 6/00

(52) U.S. Cl. CPC G01T 1/2018; A61B 6/4283; A61B 6/4233

(57) ABSTRACT

A novel X-ray detector for in-line computed tomography includes two-dimensional pixel arrays in a single piece of silicon, which allows a signal from every pixel in the silicon to be read out during an X-ray beam exposure period. The pixel arrays face the X-ray source to ensure that X-ray photons follow a straight line that intersects the X-ray source and the 2D pixel arrays. A layer of an X-ray scintillator may be applied in front of the 2D array. The detector may be implemented in a tiled detector array arrangement, or on a single PCB board. When multiple on-board detectors are arranged and mounted on a curved gantry, the novel X-ray detector system can be utilized in multi-slice in-line CT applications. The X-ray detector readout electronics is compatible with that of a conventional detector module, where signals from individual detectors can be read out in parallel.

### CROSS-REFERENCE TO RELATED APPLICATION

This application claims the benefit of U.S. Provisional Patent Application No. 62/596,136 filed Dec. 8, 2017, the contents of which are hereby incorporated by reference in its entirety.

### BACKGROUND OF THE INVENTION

The present invention generally relates to solid-state image sensors utilized in X-ray multi-slice computed tomography (CT). More specifically, the present invention relates to X-ray detectors for CT systems.

In today's modern industrial environment, a two-dimensional (2D) X-ray radiography inspection is widely utilized in a variety of industrial fields and applications. However, conventional 2D X-ray in-line radiography inspection systems have significant shortcomings in their inability to detect depth of defects, even if they can detect a location of the defects.

Computed tomography (CT) is a three-dimensional (3D) imaging technique that has been traditionally mostly utilized in medical, transportation, and personal securities applications. In recent years, the CT-based 3D imaging machines are also finding compelling applications in other industrial fields.

By analyzing a set of accumulated 3D data from a reconstruction of a matrix, which constitutes a depiction of a density function of the object section being examined, internal defects of an object can be discovered precisely at 3D coordinates. A multi-slice CT apparatus (i.e. also known as a “multi-row CT” in some instances) is particularly well-suited to achieve this task, enabling measurements of a plural number of projection data simultaneously by dividing detectors into a plural number of rows to obtain a high quality slice image. The multi-slice CT apparatus typically include an X-ray source, a fan beam, an imaging detector, and data readout electronic circuits and/or software responsible for data acquisition, image reconstruction, and image visualization.

Furthermore, in a typical multi-slice CT apparatus, an X-ray source and a detector apparatus are positioned on opposite sides of an incoming entry point of an object to be scanned and inspected. The X-ray source generates and directs an X-ray beam towards the object, while the detector apparatus measures the X-ray absorption at a plurality of transmission paths defined by the X-ray beam during the inspection process. A rotational gantry typically contains the X-ray source, the fan beam, the imaging detector, and other electrical components, wherein a mounting unit for the imaging detector usually has a curved geometry.

As the X-ray passes through an object near its center, the X-ray beam is attenuated. During an X-ray scan to acquire the X-ray projection data, gantry and other electrical components mounted inside rotate around a center of rotation in X and Y planes. Furthermore, the rotation of the gantry and the X-ray source are controlled at least in part by a main control unit of the CT apparatus, and the specific rotational speed and the position of the gantry are controlled by a gantry motor controller.

In addition, readout electronic circuits and data acquisition components provide digitized data from the multi-slice CT apparatus and transmit the digitized data to a computer. Then, an image reconstruction unit executed by the computer receives the digitized data and performs a high-speed image reconstruction. The reconstructed image is then displayed and analyzed by a monitoring personnel or an image-problem identification software to determine any defects related to the scanned object.

However, conventional multi-slice CT apparatuses tend to be substantially complicated to assemble and manufacture, resulting in higher production costs. They also tend to suffer an undesirable amount of parasitic resistance, capacitance, and/or inductance due to a multi-module assembly and complex interconnections among various modules. For example, conventional multi-slice CT configurations, as disclosed in U.S. Pat. Nos. 7,095,028 and 6,700,948 using flat panel or photodiode arrays have numerous disadvantages. First, it is difficult to make a bigger size flat panel appropriately curved. Second, photodiode arrays in the conventional multi-slice CT devices need bulky peripheral electronics. Third, when pixel sizes are scaled down to smaller dimensions, while more pixels need to be read out, a photodiode array electrical connection structure becomes more complicated.

Fourth, conventional multi-slice CT configurations are primarily optimized for low kV medical applications, and not for higher kV industrial applications. Fifth, higher kV X-rays may cause a more serious and larger object scatter problem during multi-slice data acquisitions, so more image slices of a scanned object may not necessarily yield better or higher quality images for inspection. Lastly, both manufacturing and maintenance costs of the conventional CT devices may be unnecessarily high, which can be addressed in a more efficient device structure and a related component arrangement.

Therefore, it may be desirable to devise a novel multi-slice X-ray detector structure and a related component arrangement to simplify device assembly complexities, reduce production costs, and minimize parasitic interconnection parameters. In addition, it may also be desirable to devise a novel multi-slice X-ray detector structure and a related component arrangement that provide a high-quality in-line CT image in an industrial production environment with simplified maintenance complexities and reduced maintenance costs.

### SUMMARY

Summary and Abstract summarize some aspects of the present invention. Simplifications or omissions may have been made to avoid obscuring the purpose of the Summary or the Abstract. These simplifications or omissions are not intended to limit the scope of the present invention.

The present invention is directed to a novel X-ray detector for in-line multi-slice computed tomography with a novel structural arrangement that provides improved image-scanning qualities, simpler assembly and packaging processes, lower costs of manufacturing, and/or longer operating lifetimes with improved reliability characteristics.

In a preferred embodiment of the invention, a multi-slice X-ray detector as part of a CT system has multi-element detectors arranged in a plural number of rows in an axial direction, wherein an object is to be X-ray scanned and examined while moving in a particular axial (e.g. Z-axis) direction. The X-ray is transmitted through the object by a rotating X-ray gantry including source and detector, and related energy patterns and signatures are detected by detection elements, which may be photodiodes or other photoelements. Through this process, a plural number of spiral projection data is acquired. Furthermore, the multi-slice X-ray detector also includes a radiation projection detector for generating signals in response to a radiation beam.

In the preferred embodiment of the invention, the multi-slice X-ray detector has an indirect imager that includes a conversion layer configured to generate visible light photons in response to radiation and a visible-light-sensitive photo detector array aligned with the PCB. Each photo detector array has a plurality of lines (i.e. rows, columns, or both) of detector elements, such as X-ray-detecting pixels. The ASIC unit, which is preferably integrated in the same substrate as the detector elements, can collect signals from two or more of the lines of detector elements simultaneously.

By collecting signals from two or more lines of detector elements simultaneously or in parallel, the time it takes to read signals from all lines of the detector elements in the detector can be reduced. This faster simultaneous processing, in turn, improves the frame rate of the detector. Furthermore, in one embodiment of the invention, signals from a plurality of diode chips may be retrieved from each chip simultaneously by the ASIC chip.

In one embodiment of the invention, a TDI sensor may also be utilized in the multi-slice X-ray detector for in-line computed tomography. Furthermore, MOS devices integrated into the multi-slice X-ray detector are typically radiation-shielded or radiation-resilient, and are typically located in the periphery of the pixel arrays to give sufficient spacing for dedicated radiation shielding, if necessary.

Various embodiments of the present invention is advantageous in that it simultaneously maintains good registration between the corresponding sets of pixels and resistance to radiation degradation. Other features and advantages of the present invention will be made apparent from the following detailed description and the drawings.

### BRIEF DESCRIPTION OF DRAWINGS

FIG. 1 shows a single-chip integrated multi-slice X-ray detector for in-line computed tomography, in accordance of an embodiment of the invention.

FIG. 2 shows a side-by-side two chip-aligned multi-slice X-ray detector for in-line computed tomography, in accordance with an embodiment of the invention.

FIG. 3 shows an X-ray-shielded two chip-aligned multi-slice X-ray detector for in-line computed tomography, in accordance with an embodiment of the invention.

FIG. 4 shows a top view of a tiled array of integrated multi-slice X-ray detectors on a single PCB with its associated electrical connectors, in accordance with an embodiment of the invention.

FIG. 5 shows a top view of a four PCB application assembly comprising a plurality of tiled arrays of integrated multi-slice X-ray detectors, in accordance with an embodiment of the invention.

FIG. 6 shows a perspective diagram of a CT system with a novel integrated multi-slice X-ray detector incorporated in a curved gantry and operating in a time-delay integration (TDI) mode for a quick 2D imaging, in accordance with an embodiment of the invention.

FIG. 7 shows perspective diagram of a CT system with a novel integrated multi-slice X-ray detector incorporated in a curved gantry and operating in a 3D CT mode in accordance with an embodiment of the invention.

### DETAILED DESCRIPTION

Specific embodiments of the invention will now be described in detail with reference to the accompanying figures. Like elements in the various figures are denoted by like reference numerals for consistency.

In the following detailed description of the present invention, numerous specific details are set forth in order to provide a thorough understanding of the present invention. However, it will become obvious to those skilled in the art that the present invention may be practiced without these specific details. In other instances, well known methods, procedures, and/or components have not been described in detail to avoid unnecessarily obscuring aspects of the present invention. The detailed description is presented largely in terms of procedures, logic blocks, processing, and/or other symbolic representations that directly or indirectly resemble a single-chip integrated multi-slice X-ray detector for in-line computed tomography or a method of operating thereof. These descriptions and representations are the means used by those experienced or skilled in the art to most effectively convey the substance of their work to others skilled in the art.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments mutually exclusive of other embodiments. Further, the order of blocks in process flowcharts or diagrams representing one or more embodiments of the invention does not inherently indicate any particular order nor imply any limitations in the invention.

This invention generally relates to semiconductor-implemented image sensors, which are utilized in X-ray multi-slice computed tomography (CT). More specifically, the present invention relates to X-ray detectors for CT systems.

Multi-slice detector arrays can increase the data rate by scanning a given volume with multiple parallel image slices at the same time. A helical scan is a very important in-line nonstop inspection feature in a production line, where X-ray inspection data can be taken, as the scanned object undergoes a nonstop translational motion and as the gantry rotates continuously. In order to create better resolution between slices, some scanning methods utilize an increased number of detectors in the Z-axis (axis of rotation) direction. In theory, more scan slices would result in more parallel images that can be processed rapidly.

In reality, however, there is a trade-off between number of multiple image slices and quality of each image slice. When the number of multi-slices increases, detectors behave more like a large 2D imager, which makes the X-ray scatter from a scanned object to become increasingly significant. This is especially pronounced when X-ray kV goes beyond 160 kV in an industrial environment.

Conventional detectors associated with the multi-slice CT include flat panels and 2D photodiode array configurations. A notable disadvantage of the flat panels comes from a production difficulty and a high cost to make each flat panel curved. Furthermore, the 2D photodiode arrays are typically discrete units that require bulky peripheral electronics and higher manufacturing costs.

One object of an embodiment of the present invention is to provide a novel multi-slice X-ray detector structure and a related component arrangement to simplify device assembly complexities, reduce production costs, and minimize parasitic interconnection parameters.

Another object of an embodiment of the present invention is to provide a novel multi-slice X-ray detector structure and a related component arrangement that enable a high-quality in-line CT image in an industrial production environment with simplified maintenance complexities and reduced maintenance costs.

In various embodiments of the present invention, a novel integrated semiconductor structure and a related component arrangement are utilized for devising an integrated multi-slice X-ray CT detector for in-line multi-slice CT applications. Importantly, the integrated multi-slice X-ray CT detector can utilize similar data readout strategy of a conventional LDA (Linear Diode Array) semiconductor detector.

In a conventional LDA detector, a longer-length LDA is composed of multiple groups of shorter-length LDAs, where a data readout clock is from a single source to allow synchronous readout from the multiple groups. By arranging LDA pixels into a 2D array pixel configuration while providing a data readout clock from a single source, this arrangement ensures simultaneous data readouts.

In order to increase the CT image readout refresh cycles, it is desirable to design the readout electronics to be arranged in parallel configurations. For example, it is desirable to implement an ADC readout channel per semiconductor die utilized in the readout electronics unit. In a typical industrial CT imaging application, pixel sizes may range from 100 um to 800 um, which is well within the manufacturable pixel sizes from a standard CMOS technology. Therefore, in various embodiments of the present invention, electrically-connecting a multi-slice computed tomography (CT) detector module made from the integrated CMOS arrangement to a CT system is quite simplified, compared to non-modular and non-scalable conventional detector module designs. The CMOS-integration-based multi-slice CT detector module, as disclosed in various embodiments of the present invention, provides significant cost savings in manufacturing, improved reliability, and in some cases, higher imaging performance, compared to the conventional detector device designs (e.g. LDA-based designs, etc.) that have been traditionally utilized as the multi-slice CT detector module.

Furthermore, the integrated multi-slice X-ray CT detector designed in accordance with an embodiment of the present invention can also optionally incorporate a CMOS TDI (time delay and integration) sensor with a built-in two-dimensional data readout structure. The TDI sensor is configured to perform the multi-slice CT scan functions with a corresponding CT control software module. In addition, in some embodiments of the invention, the integrated multi-slice X-ray CT detector physically separates the signal processing circuitry from the photodiode-based pixel arrays for an area-targeted X-ray shielding, wherein the signal processing circuitry is monolithically integrated on the same substrate as the pixel arrays in one embodiment, or located on a separate chip in another embodiment.

X-ray image sensors have wide applications in medical, security, industrial inspection, product quality, and product safety field. An integrated multi-slice X-ray CT detector, designed in accordance with one or more embodiments of the present invention, contains a structurally-unique arrangement of the X-ray image sensors and other electronic components that provide significant cost savings in manufacturing, improved reliability, and in some cases, higher imaging performance, compared to conventional non-scalable and non-modular detector modules in a CT system. The operating environment of the present invention is described with respect to a novel X-ray CT detector capable of executing 3D multi-slice in-line CT applications.

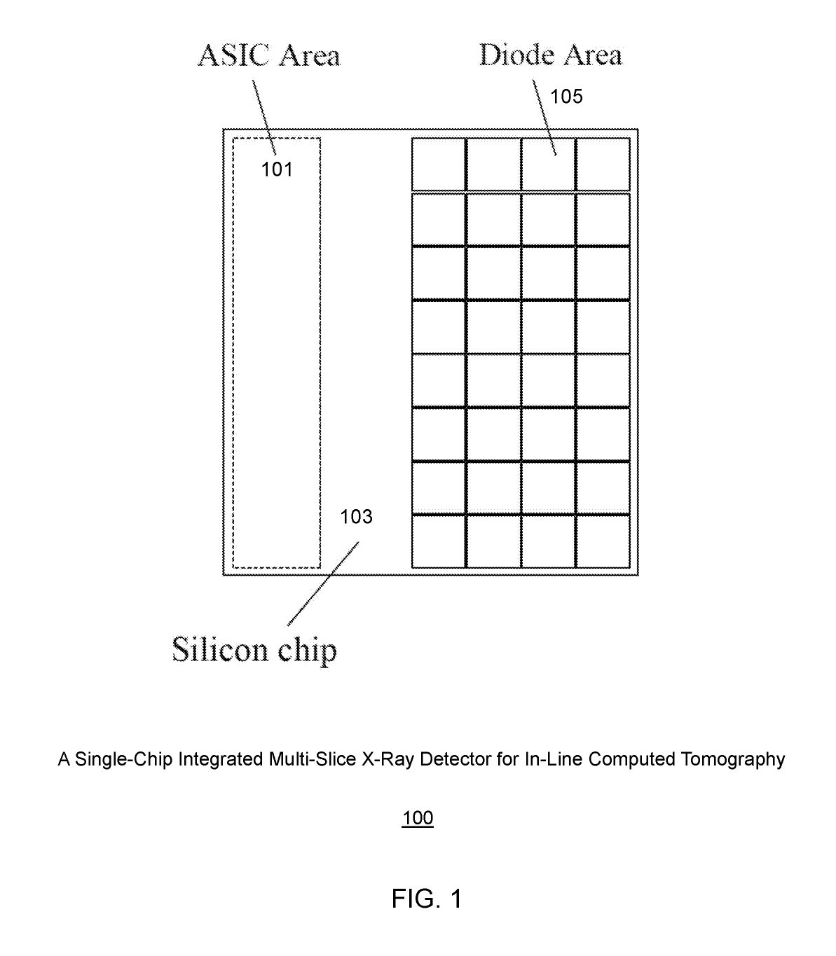

FIG. 1 shows a single-chip integrated multi-slice X-ray detector (100) that can be utilized for an in-line computed tomography (CT) system, in accordance of an embodiment of the invention. In this embodiment, one semiconductor substrate (103) (e.g. silicon, or a compound semiconductor substrate) integrates a two-dimensional (2D) pixel array (105) and an application-specific integrated circuit (ASIC) area (101). Typically, the 2D pixel array (105) is made of photodiodes or other photoelements that are capable of generating unique electrical signals correlational to specific X-ray energy levels and/or signatures through an object scanned by an X-ray emission in the in-line CT system. Such unique electrical signals generated from the 2D pixel array (105) enable signal acquisition and image reconstruction components to construct a visualized model of the scanned object. The visualized model can then be utilized by a CT system monitoring personnel to discover or analyze defects, impurities, or other notable conditions without physically disassembling the object.

Furthermore, as shown in FIG. 1, the ASIC area (101) may contain one or more electrical circuits or devices that can be utilized for signal acquisition processing, signal filtering, image reconstruction data synthesis, or any other desirable activities performed by the single-chip integrated multi-slice X-ray detector (100). Preferably, the one or more electrical circuits or devices are metal oxide semiconductor (MOS) devices, which typically include CMOS-based transistors, capacitors, and/or resistors. As shown in this embodiment, a monolithic integration of the two-dimensional (2D) pixel array (105) and the application-specific integrated circuit (ASIC) area (101) into a single semiconductor substrate (103) reduces a module footprint, manufacturing and assembly costs, and undesirable parasitic influences in signal transmission and reconstruction between the 2D pixel array (105) and signal acquisition and processing circuitry within the ASIC area (101). In some cases, this monolithic integration of the 2D pixel array (105) and the ASIC area (101) may also improve the overall reliability of the multi-slice X-ray detector, and ultimately, the device reliability of the CT system that incorporates the multi-slice X-ray detector.

Moreover, in one embodiment of the invention, an integrated multi-slice X-ray computed tomography (CT) detector (e.g. 100) includes a two-dimensional pixel array (e.g. 105) comprising rows and columns of individual pixels. The two-dimensional pixel array is designed to transmit information stored in the individual pixels as electrical signals to one or more MOS devices in an ASIC area (e.g. 101), when the two-dimensional pixel array is exposed to an X-ray emission for a prespecified amount of time. Moreover, in one embodiment of the invention, the integrated multi-slice X-ray computed tomography (CT) detector may monolithically integrate the 2D pixel array and the ASIC area in one semiconductor substrate (e.g. 103).

The integrated multi-slice X-ray computed tomography (CT) detector also includes a metal semiconductor oxide (MOS) device located in a periphery of the two-dimensional pixel array. Preferably, the MOS device is also integrated monolithically on the single semiconductor substrate with the two-dimensional pixel array and an X-ray shielding element above or below the MOS device. In addition, the X-ray shielding element sufficiently blocks off or absorbs the X-ray emission from directly impinging on the MOS device located around the periphery of the two-dimensional pixel array. In this case, a straight-line X-ray path emanating from an X-ray source intersects the individual pixels in the two-dimensional pixel array simultaneously and does not impinge on the MOS device under the X-ray shielding element.

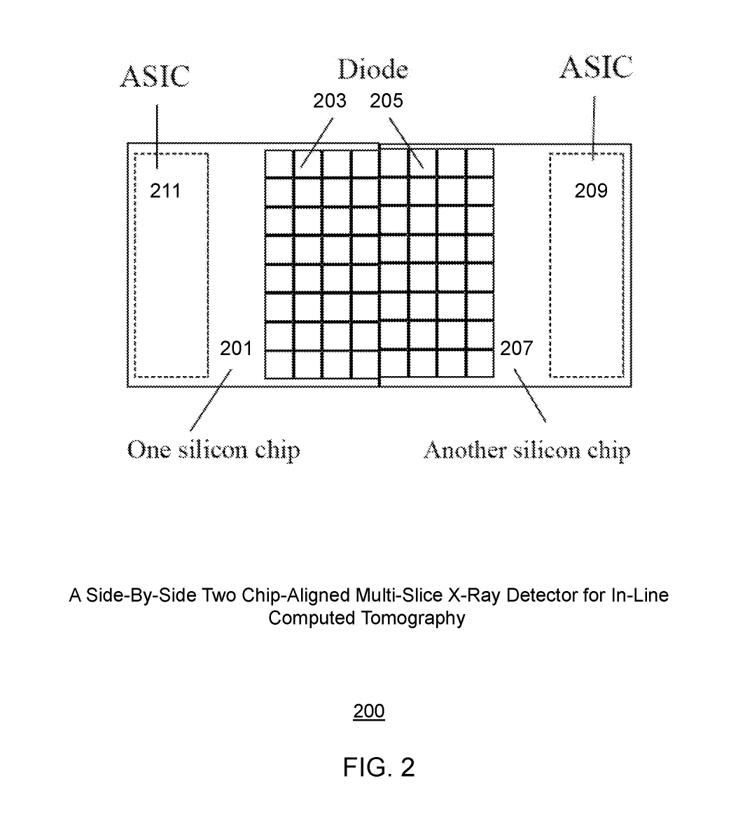

FIG. 2 shows a side-by-side two chip-aligned multi-slice X-ray detector (200) for in-line computed tomography, in accordance with an embodiment of the invention. In this embodiment, a first semiconductor substrate (201) (e.g. silicon, compound semiconductor, etc.) integrates a first two-dimensional (2D) pixel array (203) and a first application-specific integrated circuit (ASIC) area (211), while a second semiconductor substrate (207) integrates a second two-dimensional (2D) pixel array (205) and a second ASIC area (209), as shown in FIG. 2.

In industry applications where a larger detection area or a larger number of image slices are needed, it may be advantageous to create a combination of pixel arrays by adjoining the two 2D pixel arrays (203, 205), which are made of photodiodes or other photoelements to generate unique electrical signals correlating to specific X-ray energy levels and/or signatures through an object scanned by an X-ray emission in the in-line CT system. Such unique electrical signals generated from the 2D pixel array pair (203, 205) enable signal acquisition and image reconstruction components to construct a visualized model of the scanned object. The visualized model can then be utilized by a CT system monitoring personnel to discover or analyze defects, impurities, or other notable conditions without physically disassembling the object.

In this embodiment of the invention, each array in the adjoined 2D pixel array pair (203, 205) has its own ASIC area (211 or 209) that contains one or more electrical circuits or devices that can be utilized for signal acquisition processing, signal filtering, image reconstruction data synthesis, or any other desirable activities performed by the side-by-side two chip-aligned multi-slice X-ray detector (200). Preferably, each ASIC area (211 or 209) contains MOS devices for signal acquisitions, processing, filtering, and/or image reconstruction from electrical signals transmitted from its side of the adjoined 2D pixel array pair (203, 205).

For example, a first set of MOS devices located in the first ASIC area (211) may execute signal acquisitions, processing, filtering, and/or image reconstruction from the electrical signals transmitted from the first 2D pixel array (203), while a second set of MOS devices located in the second ASIC area (209) may execute signal acquisitions, processing, filtering, and/or image reconstruction from the electrical signals transmitted from the second 2D pixel array (205). In a preferred embodiment of the invention, an image reconstruction module located either within or outside of the side-by-side two chip-aligned multi-slice X-ray detector (200) is able to combine or stitch two sets of electrical signals from the adjoined 2D pixel array pair (203, 205) to generate a total set of CT images of the object scanned in the large detection area adjoined by the two independent chips. Furthermore, the large detection area formed by the adjoined 2D pixel arrays also enables the side-by-side two chip-aligned multi-slice X-ray detector (200) to maximize the number of image slices.

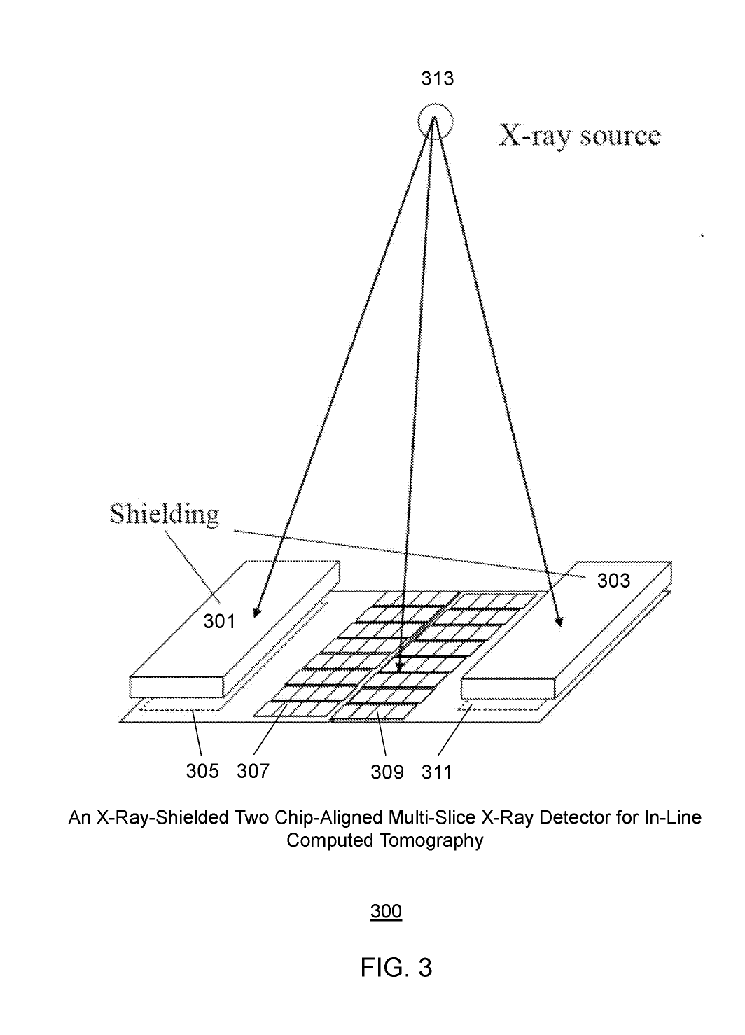

FIG. 3 shows an X-ray-shielded two chip-aligned multi-slice X-ray detector (300) for in-line computed tomography, in accordance with an embodiment of the invention. In this embodiment, a first semiconductor substrate integrates a first two-dimensional (2D) pixel array (307) and a first application-specific integrated circuit (ASIC) area (305) protected by a first radiation shield (301), while a second semiconductor substrate integrates a second two-dimensional (2D) pixel array (309) and a second ASIC area (311) protected by a second radiation shield (303), as shown in FIG. 3.

As shown in this embodiment of the invention, the X-ray-shielded two chip-aligned multi-slice X-ray detector (300) receives an X-ray emission from an X-ray source (313) located above the 2D pixel array pair (307, 309). Each of the first radiation shield (301) and the second radiation shield (303) is an X-ray shielding element that sufficiently attenuates the X-ray emission from directly impinging on a metal semiconductor oxide (MOS) device integrated in each ASIC area (305 or 311), which is typically located in a periphery of the 2D pixel array (307 or 309). As illustrated in FIG. 3, a straight-line X-ray path emanating from the X-ray source (313) intersects individual pixels in each of the two-dimensional pixel arrays (307, 309) simultaneously and does not impinge on the MOS device under the X-ray shielding element.

In industry applications where a larger detection area or a larger number of image slices are needed, it may be advantageous to create a combination of pixel arrays by adjoining the two 2D pixel arrays (307, 309), which are made of photodiodes or other photoelements to generate unique electrical signals correlating to specific X-ray energy levels and/or signatures through an object scanned by an X-ray emission in the in-line CT system. Such unique electrical signals generated from the 2D pixel array pair (307, 309) enable signal acquisition and image reconstruction components to construct a visualized model of the scanned object. The visualized model can then be utilized by a CT system monitoring personnel to discover or analyze defects, impurities, or other notable conditions without physically disassembling the object.

In this embodiment of the invention, each array in the adjoined 2D pixel array pair (307, 309) has its own ASIC area (305 or 311) that contains one or more electrical circuits or devices that can be utilized for signal acquisition processing, signal filtering, image reconstruction data synthesis, or any other desirable activities performed by the X-ray-shielded two chip-aligned multi-slice X-ray detector (300). Preferably, each ASIC area (305 or 311) contains MOS devices for signal acquisitions, processing, filtering, and/or image reconstruction from electrical signals transmitted from its side of the adjoined 2D pixel array pair (307, 309).

In this embodiment of the invention, an image reconstruction module located either within or outside of the X-ray-shielded two chip-aligned multi-slice X-ray detector (300) is able to combine or stitch two sets of electrical signals from the adjoined 2D pixel array pair (307, 309) to generate a total set of CT images of the object scanned in the large detection area adjoined by the two independent chips. Furthermore, the large detection area formed by the adjoined 2D pixel arrays also enables the X-ray-shielded two chip-aligned multi-slice X-ray detector (300) to maximize the number of image slices.

Furthermore, a preferred embodiment of the invention, pixel elements within each pixel does not contain electronic components that are easily susceptible to X-ray radiation damage. Moreover, MOS devices are typically placed sufficiently apart from the 2D pixel arrays to facilitate the construction of X-ray shields, which are made of a dense and high-impedance (Z) material capable of absorbing or deflecting a majority of incident X-ray photons within its shield structure.

In addition, the MOS devices around the borders of the pixel arrays enable the 2D pixel arrays to be read-out to external devices through an image sensor interface. Typically, these MOS devices include pixel amplifiers. They can also include gain amplifiers, noise-cancellation circuitry, signal-chain circuitry, output drivers, timing circuitry with digital scanning shift registers for selecting a series of pixels, and biasing circuitry.

An advantage of utilizing CMOS circuitry for MOS devices in the above embodiments is that standard CMOS processes can be utilized to lower manufacturing costs, compared to higher device production costs for conventional non-modular and non-scalable detector designs. Furthermore, in case of CMOS device manufacturing, radiation-hardened CMOS processes are widely available. In some embodiments of the invention, pixels can include scintillators and silicon photodiodes and have associated silicon CMOS circuitry. Alternative embodiments may utilize other semiconductor circuitry without silicon photodiodes or CMOS circuitry. For example, in such alternative embodiments, photoelements that work with direct X-ray radiation can be utilized instead, without the need for scintillators.

It is noted herein that the pixel arrays are typically implemented in planar structures, but the arrays do not have to be rectilinear. They can also be circular, linear, or any number of arbitrary shapes. It is preferable that the pixel arrays do not contain MOS devices within the pixel arrangement themselves to avoid radiation-related weaknesses. Therefore, it is desirable to place the MOS devices in an ASIC area with a dedicated X-ray shield.

In one embodiment of the invention, an integrated multi-slice X-ray computed tomography (CT) detector includes a two-dimensional pixel array comprising rows and columns of individual pixels. The two-dimensional pixel array is able to transmit information stored in the individual pixels as electrical signals, when the two-dimensional pixel array is exposed to an X-ray emission for a prespecified amount of time. Preferably, the individual pixels in the two-dimensional pixel array are monolithically integrated in a single semiconductor substrate.

Furthermore, the integrated multi-slice X-ray computed tomography (CT) detector also includes a metal semiconductor oxide (MOS) device located around a periphery of the two-dimensional pixel array. The MOS device is at least one of a signal acquisition circuitry and a time delay and integration (TDI) circuitry that can provide a time delay and summing to generate combined electrical signals in a line-scanning format that contain two-dimensional object scan information. Preferably, the MOS device is also integrated monolithically on the single semiconductor substrate with the two-dimensional pixel array and an X-ray shielding element above or below the MOS device.

In this embodiment, the X-ray shielding element is able to sufficiently absorb or deflect the X-ray emission from directly impinging on the MOS device located around the periphery of the two-dimensional pixel array. A straight-line X-ray path emanating from an X-ray source intersects the individual pixels in the two-dimensional pixel array simultaneously and does not impinge on the MOS device under the X-ray shielding element.

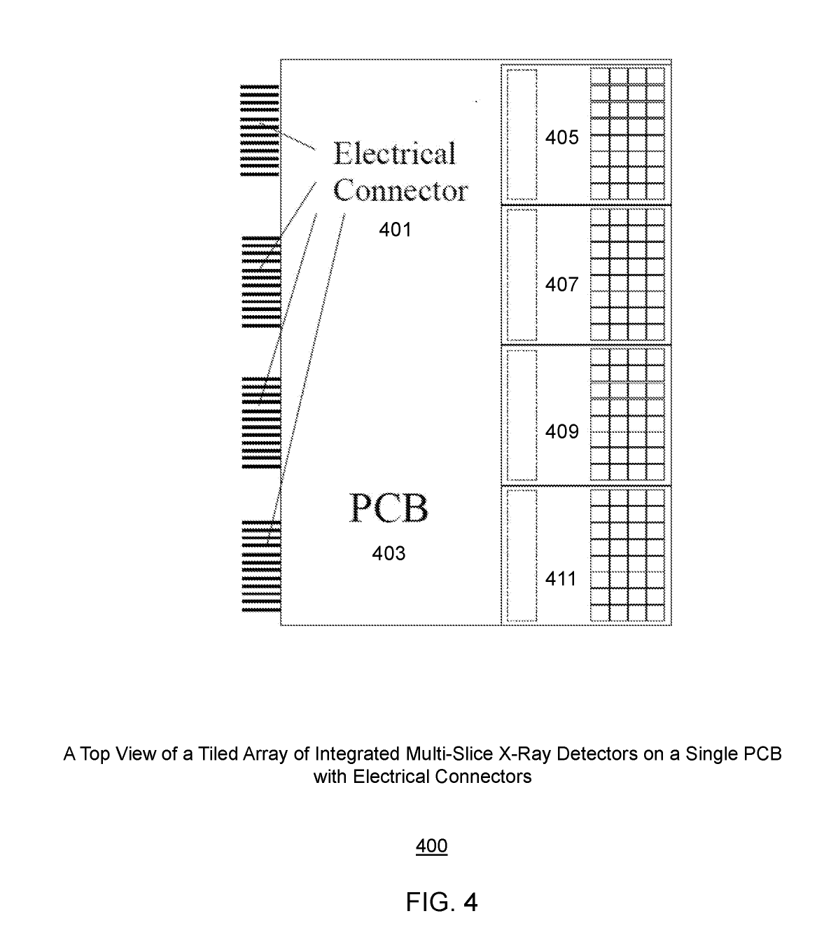

FIG. 4 shows a top view of a novel and uniquely-tiled array of integrated multi-slice X-ray detectors (400) on a single PCB (403) with its associated electrical connectors (401), in accordance with an embodiment of the invention. In this embodiment, four individually-functioning integrated multi-slice X-ray detectors (405, 407, 409, 411) constitute the tiled array connected to the single PCB (403). The consolidated electrical connectors (401) in the single PCB (403) enable the CT image sensor signals acquired and processed within the tiled array and the single PCB (403) to transmit image-related electrical signals to another part of the CT system, without undesirable interconnection wiring clutters commonly present in conventional CT detector modules.

The tiled array configuration, as shown in FIG. 4, allows the CT system to accommodate a large, scalable, and modular number of 2D pixel arrays that can generate an increased number of image slices and a larger detection area. The ease of modularity and scalability of the 2D pixel arrays in this novel tiled configuration to custom-design and custom-define detector areas and CT image slice capabilities rapidly and inexpensively without a major redesign of the detector module, depending on particular needs of an industrial application, is one of the key aspects of the novelty of the present invention.

Even though only four individually-functioning integrated multi-slice X-ray detectors (405, 407, 409, 411) are shown in this embodiment to constitute the tiled array, the number of integrated multi-slice X-ray detectors utilized to form the tiled array may be scaled up or down in another embodiment, depending on the needs of particular performance parameters of the CT system.

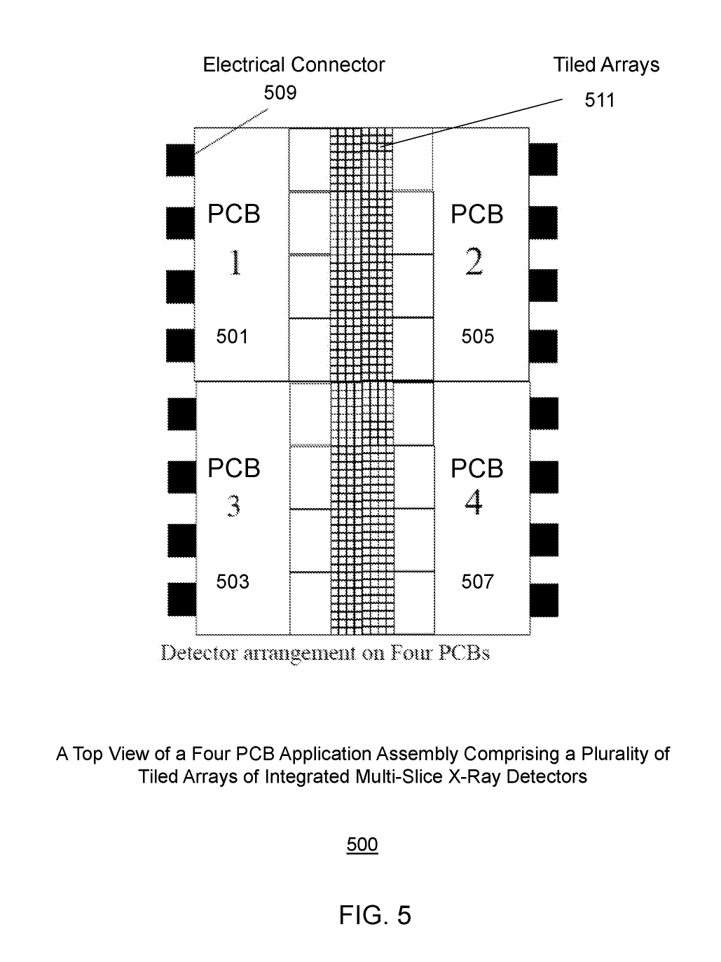

FIG. 5 shows a top view of a four PCB application assembly (500) comprising a plurality of tiled arrays (511) of integrated multi-slice X-ray detectors, in accordance with an embodiment of the invention. In this embodiment, four PCBs (501, 503, 505, 507) each contain four individually-functioning integrated multi-slice X-ray detectors to constitute the plurality of tiled arrays (511) with a total of sixteen individually-functioning integrated multi-slice X-ray detectors, as shown in FIG. 5.

The electrical connectors (509) for the four PCBs (501, 503, 505, 507) are located on an outside boundary of each PCB for ease of electrical connections to other components of a CT system. Typically, CT image sensor signals acquired and processed within the plurality of tiled arrays (511) and the four PCBs (501, 503, 505, 507) are transmitted to other parts of the CT system.

The plurality of tiled arrays (511) configuration, as shown in FIG. 5, allows the CT system to accommodate a large number of 2D pixel arrays that can generate an increased number of image slices and a larger detection area. This uniquely-tiled array configuration (i.e. 500, 511), as shown in FIG. 5, allows the CT system to accommodate a large, scalable, and modular number of 2D pixel arrays that can generate an increased number of image slices and a larger detection area. The ease of modularity and scalability of the 2D pixel arrays in this novel tiled configuration (i.e. 500, 511) to custom-design and custom-define detector areas and CT image slice capabilities rapidly and inexpensively without a major redesign of the detector module, depending on particular needs of an industrial application, is one of the key aspects of the novelty of the present invention.

Even though sixteen individually-functioning integrated multi-slice X-ray detectors connected to four PCBs are shown in this embodiment to constitute the plurality of tiled arrays (511), the number of integrated multi-slice X-ray detectors utilized to form the tiled arrays may be scaled up or down in another embodiment, depending on the needs of particular performance parameters of the CT system.

In a multi-slice CT system, the integrated multi-slice X-ray detectors, as described in various embodiments of the invention, can be arranged in a curved geometry of a gantry installed in the multi-slice CT system. Mounting a multi-slice X-ray detector board in the curved geometry of the gantry is nearly as seamless as mounting a conventional-design detector device into the multi-slice CT system. Furthermore, the integrated multi-slice X-ray detectors can reduce cabling requirements with the rest of the multi-slice CT system, compared to the conventional detector device designs that traditionally pose more challenges for interconnections within the multi-slice CT system.

In various embodiments of the invention, several smaller pixel sub-arrays can be tiled or butted together to create the full pixel arrays. For the tiled arrangement as shown in FIG. 4 and FIG. 5, it is desirable to minimize the gap between tiles to provide a uniform array of pixels. The accurate registration of pixels in the above embodiments does not preclude the use of software algorithms that improve the registration between corresponding pixels. Having accurate physical registration improves the performance of software algorithms and reduces the significance of tradeoffs, such as loss of image resolution, sharpness, and fidelity.

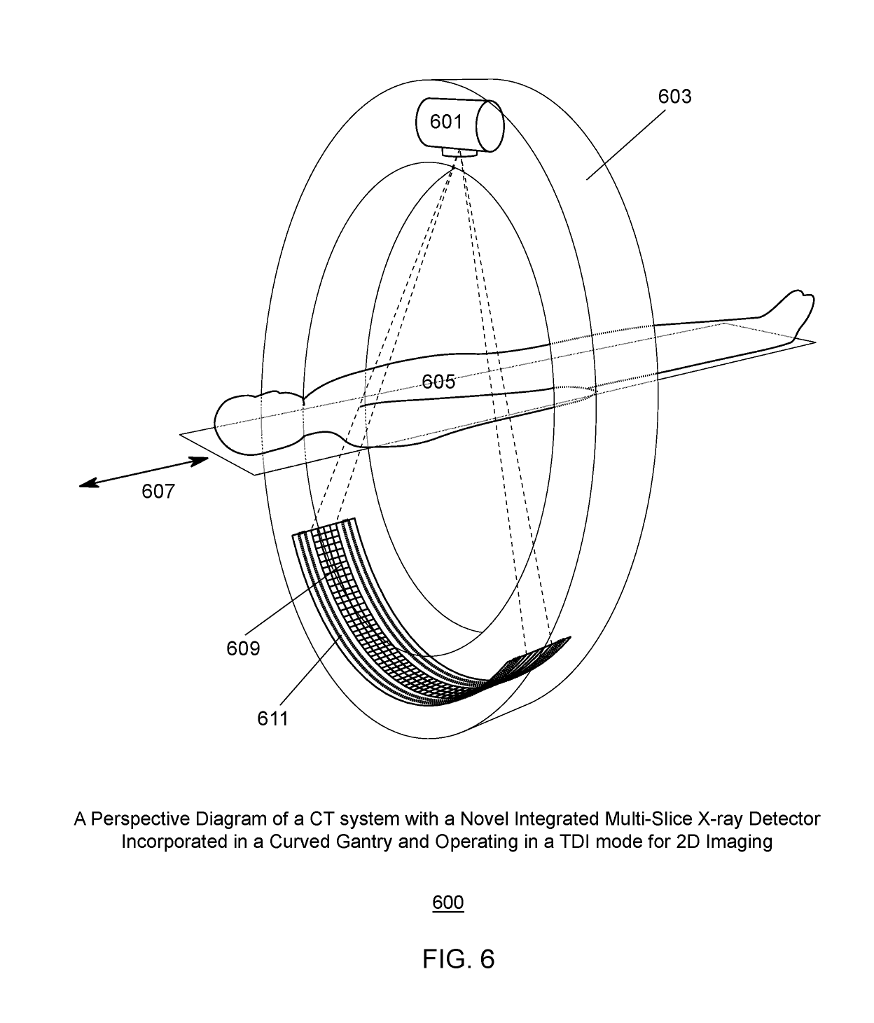

FIG. 6 shows a perspective diagram (600) of a CT system with a novel integrated multi-slice X-ray detector (i.e. 609, 611) incorporated in a curved gantry (603) and operating in a time-delay integration (TDI) mode for a quick 2D imaging, in accordance with an embodiment of the invention. As shown in this perspective diagram (600), the integrated multi-slice X-ray detector, including 2D pixel arrays (609) and MOS devices in an ASIC area (611), is monolithically integrated in one chip and together with an X-ray shielding element for the MOS devices is contained within the curved gantry (603), in a preferred embodiment of the invention. In another embodiment of the invention, the integrated multi-slice X-ray detector (i.e. 609, 611) may further utilize the uniquely-modular, scalable, and novel adjoining assembly structures (i.e. described in FIG. 4 or FIG. 5) that either modularly add or subtract a desirable number of independently-functioning individual units of integrated multi-slice X-ray detectors (i.e. as described in FIG. 1) to increase, decrease, or custom-define the detector area and image slicing capabilities. Furthermore, electrical connectors can be placed on the outside boundaries of the integrated multi-slice X-ray detector (i.e. 609, 611) for interconnection with other modules within the CT system, as previously illustrated in FIG. 4 and FIG. 5.

In this embodiment of the invention, the CT system operates in a time-delay integration (TDI) mode for a quick 2D imaging, which does not require the curved gantry (603) to rotate around a subject (605) under a scanning process with X-ray emissions from an X-ray source (601). Typically, only the subject (605) is required to move along a Z-axis (607), as illustrated in FIG. 6, to generate the quick 2D imaging in the TDI mode, which enables a fast and exploratory 2D digital radiographic image to determine whether a more time-consuming 3D CT scan is necessary. The quick 2D imaging also minimizes unnecessarily-prolonged radiation exposures to the subject (605). In some instances, the quick 2D imaging enabled by the TDI mode also assists in identifying a location of particular interest prior to a full 3D CT scan to prepare and optimize imaging scan parameters to minimize wasted time and error.

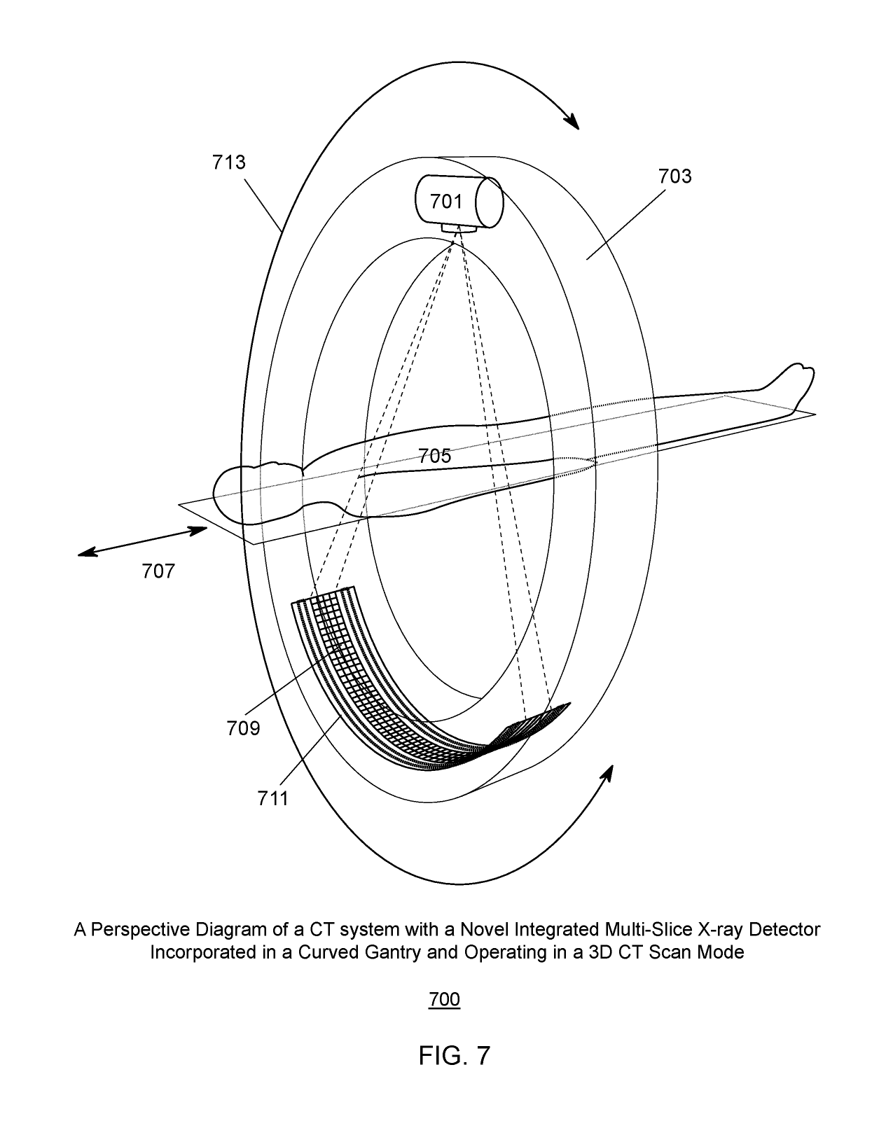

FIG. 7 shows perspective diagram (700) of a CT system with a novel integrated multi-slice X-ray detector (i.e. 709, 711) incorporated in a curved gantry (703) and operating in a 3D CT mode in accordance with an embodiment of the invention. As shown in this perspective diagram (700), the integrated multi-slice X-ray detector, including 2D pixel arrays (709) and MOS devices in an ASIC area (711), is monolithically integrated in one chip and together with an X-ray shielding element for the MOS devices is contained within the curved gantry (703), in a preferred embodiment of the invention. In another embodiment of the invention, the integrated multi-slice X-ray detector may further utilize the uniquely-modular, scalable, and novel adjoining assembly structures (i.e. described in FIG. 4 or FIG. 5) that either modularly add or subtract a desirable number of independently-functioning individual units of integrated multi-slice X-ray detectors (i.e. as described in FIG. 1) to increase, decrease, or custom-define the detector area and image slicing capabilities.

In this embodiment of the invention, the CT system operates in a full 3D CT scan mode that typically involves a Z-axis movement (707) of a subject (705) undergoing the scan with X-ray emissions from an X-ray source (701) and a rotational movement (713) of the curved gantry (703) to capture three-dimensional multiple image slices of the subject (705). In a preferred embodiment of the invention, the full 3D CT scan is typically utilized, if a quick 2D imaging from the TDI mode provided by the integrated multi-slice X-ray detector merits further investigation or probing of the subject (705), which may be a human or another specimen undergoing inspection or analysis.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. An integrated multi-slice X-ray computed tomography (CT) detector comprising:
a two-dimensional pixel array comprising rows and columns of individual pixels, wherein the two-dimensional pixel array is configured to transmit information stored in the individual pixels as electrical signals, when the two-dimensional pixel array is exposed to an X-ray emission for a prespecified amount of time, wherein the individual pixels in the two-dimensional pixel array are monolithically integrated in a single semiconductor substrate;
a metal semiconductor oxide (MOS) device also monolithically integrated in a periphery of the two-dimensional pixel array in the single semiconductor substrate; and
an X-ray shielding element located above or below the MOS device, wherein the X-ray shielding sufficiently attenuates the X-ray emission from directly impinging on the MOS device located in the periphery of the two-dimensional pixel array, and wherein a straight-line X-ray path emanating from an X-ray source intersects the individual pixels in the two-dimensional pixel array simultaneously and does not impinge on the MOS device under the X-ray shielding element.

2. The integrated multi-slice X-ray computed tomography (CT) detector of claim 1, further comprising a signal acquisition circuitry and a time delay and integration (TDI) circuitry that provide a time delay and summing to output in a line-scanning format for generating two-dimensional scans, and wherein the signal acquisition circuitry and the TDI circuitry are integrated into the MOS device.

3. The integrated multi-slice X-ray computed tomography (CT) detector of claim 1, wherein the MOS device is a CMOS silicon chip.

4. The integrated multi-slice X-ray computed tomography (CT) detector of claim 1, wherein the individual pixels in the two-dimensional array contain photodiodes or photoelements resistant to radiation damage.

5. The integrated multi-slice X-ray computed tomography (CT) detector of claim 1, wherein the individual pixels in the two-dimensional pixel array contain scintillator materials that convert X-ray photons to lower-energy or to visible photons, which are readily detectable by photodiodes.

6. The integrated multi-slice X-ray computed tomography (CT) detector of claim 1, wherein the MOS device located in the periphery of the two-dimensional pixel array is a CMOS field-effect transistor or capacitor.

7. The integrated multi-slice X-ray computed tomography (CT) detector of claim 1, wherein the two-dimensional pixel array comprises multiple semiconductor substrates, each with a portion of a plurality of photodiodes or photoelements that make up the two-dimensional pixel array, wherein the multiple semiconductor substrates are arranged tightly in a tile formation to constitute a complete version of the two-dimensional pixel array.

8. The integrated multi-slice X-ray computed tomography (CT) detector of claim 1, wherein no visible-light optics are utilized for a multi-slice X-ray CT application.

9. The integrated multi-slice X-ray computed tomography (CT) detector of claim 1, wherein a curved gantry mounting one or two rows of printed circuit boards (PCBs) containing a plurality of two-dimensional pixel arrays provides a multi-slice or multi-row CT detector configuration.

10. An integrated multi-slice X-ray computed tomography (CT) detector comprising:
a two-dimensional pixel array comprising rows and columns of individual pixels, wherein the two-dimensional pixel array is configured to transmit information stored in the individual pixels as electrical signals, when the two-dimensional pixel array is exposed to an X-ray emission for a prespecified amount of time, wherein the individual pixels in the two-dimensional pixel array are monolithically integrated in a single semiconductor substrate;
a metal semiconductor oxide (MOS) device also monolithically integrated in a periphery of the two-dimensional pixel array in the single semiconductor substrate, wherein the MOS device contains a signal acquisition circuitry and a time delay and integration (TDI) circuitry that provide a time delay and summing to output in a line-scanning format for generating two-dimensional scans; and
an X-ray shielding element located above or below the MOS device, wherein the X-ray shielding sufficiently attenuates the X-ray emission from directly impinging on the MOS device located in the periphery of the two-dimensional pixel array, and wherein a straight-line X-ray path emanating from an X-ray source intersects the individual pixels in the two-dimensional pixel array simultaneously and does not impinge on the MOS device under the X-ray shielding element.

11. The integrated multi-slice X-ray computed tomography (CT) detector of claim 10, wherein the MOS device is a CMOS silicon chip.

12. The integrated multi-slice X-ray computed tomography (CT) detector of claim 10, wherein the individual pixels in the two-dimensional array contain photodiodes or photoelements resistant to radiation damage.

13. The integrated multi-slice X-ray computed tomography (CT) detector of claim 10, wherein the individual pixels in the two-dimensional pixel array contain scintillator materials that convert X-ray photons to lower-energy or to visible photons, which are readily detectable by photodiodes.

14. The integrated multi-slice X-ray computed tomography (CT) detector of claim 10, wherein the MOS device located in the periphery of the two-dimensional pixel array is a CMOS field-effect transistor or capacitor.

15. The integrated multi-slice X-ray computed tomography (CT) detector of claim 10, wherein the two-dimensional pixel array comprises multiple semiconductor substrates, each with a portion of a plurality of photodiodes or photoelements that make up the two-dimensional pixel array, wherein the multiple semiconductor substrates are arranged tightly in a tile formation to constitute a complete version of the two-dimensional pixel array.

16. The integrated multi-slice X-ray computed tomography (CT) detector of claim 10, wherein no visible-light optics are utilized for a multi-slice X-ray CT application.

17. The integrated multi-slice X-ray computed tomography (CT) detector of claim 10, wherein a curved gantry mounting one or two rows of printed circuit boards (PCBs) containing a plurality of two-dimensional pixel arrays provides a multi-slice or multi-row CT detector configuration.
